(19) United States
(12) Patent Application Publication
(10) Pub. No.: US 2014/0100551 A1
(43) Pub. Date: Apr. 10, 2014

(54) FLUSHING DEVICE AND A CATHETER KIT COMPRISING A FLUSHING DEVICE

(71) Applicant: Christopher Holmstrom, Vasteras (SE)

(72) Inventor: Christopher Holmstrom, Vasteras (SE)

(73) Assignee: CATHEASY VASTERAS AB, Vasteras (SE)

(21) Appl. No.: 14/123,263

(22) PCT Filed: Jun. 1, 2012
(86) PCT No.: PCT/SE2012/050586

§ 371 Date: Dec. 2, 2013

(30) Foreign Application Priority Data
Jun. 1, 2011 (SE) 1150513-8

(51) Int. Cl. A61M 25/01; A61M 3/02

(52) U.S. Cl. CPC A61M 25/01; A61M 3/0262
USPC 604/517; 604/403; 604/181

(57) ABSTRACT

A flushing device (8) usable in the catheterization of a bladder includes a flexible container (9) defining a compartment (12) adapted to be filled with a liquid, and a connector (10) attached to the container (9). The connector (10) is adapted to be connected to a catheter, and the connector (10) is provided with an opening (11). The flexible container (9) is interconnected with the connector (10) via a lumen (13), such that liquid is allowed to pass through the connector (10) in the lumen (13) from the flexible container (9) and exit through the opening (11). The flexible container further includes a non-return valve (14) arranged in the lumen (13). A kit including such flushing device (8) is also described.

### FIELD OF THE INVENTION

The present invention relates to a flushing device for catheterizing the bladder and a kit comprising a flushing device.

### BACKGROUND

Sometimes a catheter must be used for draining urine from the bladder for collection, or to inject liquids used for treatment or diagnosis of bladder conditions. A catheter is a flexible tube that is passed through the urethra and into the bladder. In a conventional catheter, the tube has two separated channels, or lumens, running down its length. One lumen is open at both ends, and allows urine to drain out into a collection bag. The other lumen has a valve on the outside end and connects to a balloon at the tip, the balloon is inflated when it is positioned inside the bladder, in order to stop it from slipping out.

Introducing a catheter for catheterizing the bladder may be difficult, and especially for male patients it can be a painful procedure. The procedure oftentimes causes tissue damage or bleeding, which can lead to serious infections, and it sometimes causes cramps and spasticity resulting in even more difficulties in introducing a catheter.

Today, in hospitals the usual method for catheterizing the bladder, taken from “The handbook of health care”, Västra Götalands region is performed as follows:
1. A sterile gel/anaesthetic gel is applied to the tip of a catheter and is also introduced into the urethra and allowed to act for five minutes (20 g of gel is a suitable dose for male patients).
2. The penis is lifted to a position in which it is perpendicular to the patient's body.
3. The tube is placed against the opening of the urethra and half of the gel is slowly squeezed in. A resistance will be felt when the gel reaches the sphincter.
4. Wait, while exerting pressure over the end of the penis, or use a penis clamp.
5. After approximately 1 minute, when the sphincter relaxes, the remaining amount of gel is introduced. In this way the whole urethra is filled with gel.
6. The catheter is fed in carefully into the urethra, directly out of the packaging. Alternatively one can take out the catheter from the packaging and place it in a kidney dish and introduce it using sterile or clean tweezers or with gloves. Never force it.
7. The foreskin is retracted and the penis held outstretched.
8. The entire length of the catheter is introduced.
9. Wait for a spontaneous flow of urine. Allow the urine to run out into a clean kidney dish.
10. The catheter balloon is filled with the amount of fluid recommended by the manufacturer, usually 10 ml of sterile water.

An advantage with the above described procedure is that it is a well-proven method. However, there are disadvantages, i.e. it can cause cramps, pain, bleeding and tissue damage, which in turn increases the risk for infection on catheterizing patients with enlarged prostates, neurological diseases etc. Thus, there is a need for an improved procedure for introducing a catheter.

The present invention aims to solve the problems of the conventional method for introducing a catheter.

### SUMMARY OF THE INVENTION

An object of the present invention is thus to provide an improved procedure for catheterization.

The above-mentioned object is achieved by the present invention according to the independent claims.

Preferred embodiments are set forth in the dependent claims.

Thus, in a first aspect of the invention there is provided a flushing device usable in the catheterization of a bladder. The flushing device comprises a flexible container defining a compartment adapted to be filled with a liquid, a connector attached to the container, the connector being adapted to be connected to a catheter, and said connector being provided with an opening. The flexible container is interconnected with said connector via a lumen, such that liquid can be passed through said connector in said lumen from said flexible container and exit said opening. The flushing device further comprises a non-return valve arranged in said lumen.

In order to eliminate or at least reduce the drawbacks with the prior art method, the inventor has devised a new method which uses the new and inventive device.

An advantage with the novel method over the prior art method is that the catheter does not cause cramps when it is inserted together with liquid from the flushing device. Also, tissue damage and bleeding do not occur with this gentle method. The urethra does not need to be filled with anaesthetic lubricant, reducing available space in the lumen of the urethra, as is the case with normal catheters.

Patients with restrictions in the urethra or with enlarged prostates are a group in which one can experience technical problems in introducing a catheter. The lumen of the urethra is smaller due to restrictions or prostate enlargement and the area thus becomes difficult to penetrate with the catheter. With the flushing method described here, the catheter quite simply travels past any restrictions together with the liquid.

The method provides for gentle and pain-free introduction of the catheter accompanied by minimal risk for tissue damage or bleeding which can lead to serious infections.

The method is particularly suitable for neurologically handicapped patients, who have an increased tendency to suffer cramps/spasticity resulting in difficulties in introducing a catheter. Cramps are not induced when using my method as the procedure is rapid and does not irritate the tissue.

One does not need to change the position of the penis in order to fill the urethra with anaesthetic gel, an action which can itself induce cramps. The need for a specialist (urologist) to carry out catheter insertion is reduced.

Furthermore, the method saves time since one does not need to wait for an anaesthetic to take effect. One does not either need to use expensive, specialised catheters. Above all, the method is painless which makes it both patient- and user-friendly.

### SHORT DESCRIPTION OF THE APPENDED DRAWINGS

FIG. 1 shows a schematic view of a catheter according to the prior art.

FIG. 2 shows an elevated view of the flushing device for catheterizing the bladder according to the present invention.

FIG. 3 shows a side view of the flushing device for catheterizing the bladder according to the present invention.

FIG. 4 shows a front view of the flushing device for catheterizing the bladder according to the present invention.

FIG. 5 shows a sectional view of the flushing device and the non-return valve in detail, according to one embodiment of the present invention.

### DETAILED DESCRIPTION OF PREFERRED EMBODIMENTS OF THE INVENTION

For better understanding of the context in which the flushing device is going to be used, a schematic view of a conventional catheter 1 for catheterizing the bladder is shown in FIG. 1. The catheter 1 comprises a flexible tube 2 that is adapted to pass through the urethra and into the bladder. The tube 2 comprises two separated channels, or lumens, running down its length. A first lumen is open at both ends 3′, 3″, and allows urine to drain out, the second lumen has a valve 4 on the outside end 5 and is in fluid communication with a balloon 6 at the tip 7.

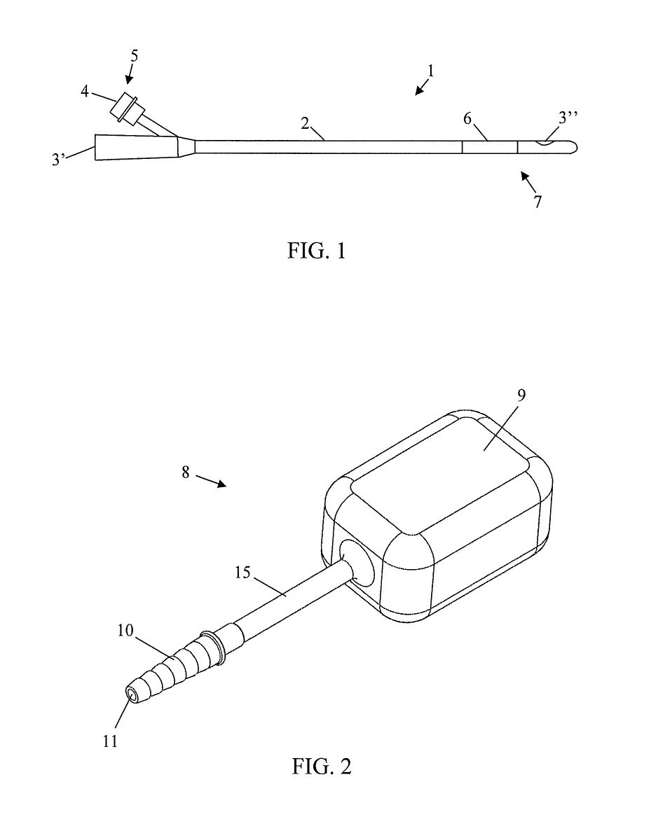

FIG. 2 shows an embodiment of a flushing device 8 usable in the catheterization of the bladder. The flushing device 8, comprises a flexible container 9 defining a compartment 12 (not shown in FIG. 2) adapted to be filled with a liquid, and a connector 10 provided with an opening 11. The connector 10 is adapted to be connected to a catheter (not shown). The flexible container 9 is interconnected with the connector 10 via a tube 15 exhibiting an inner lumen 13 (see FIG. 5), such that liquid is allowed to pass through the lumen 13 from the flexible container 9 to the opening 11, and a non-return valve 14 is arranged in the lumen 13.

In use, the flushing device 8 is connected to a drainage outlet 3′ of a catheter for catheterizing the bladder, see FIG. 1. During insertion of the catheter, the operator exerts pressure on the flexible container 9, such that liquid flows out of the compartment 12, through the tube 15, and exits through the opening 11 in the connector 10, and flows into the catheter. The liquid then passes through the catheter lumen to the opening 3″ at the catheter tip. During the insertion of the catheter, the liquid, flushed from the flushing device 8 through the catheter, acts as a lubricant. The urethra may also be moistened with liquid before starting the procedure and thereafter the catheter inserted. During insertion, the operator preferably presses on the flexible container 9 2-3 times.

The compartment 12 is preferably filled with water or a physiologocal sodium chloride solution. The compartment 12 may be pre-filled or connected to a liquid source via a tubing (not shown). Perferably, the compartment 12 contains about 100 ml liquid. In one embodiment, the compartment 9 contains 100 ml NaCl 9 mg/ml, or alternatively 100 ml NaCl 9 mg/ml with the addition of Xylocaine 10 mg/ml for anaesthetic purposes. The flexible container 9 may further be provided with a valve (not shown) such that air is allowed to pass into the compartment 12, such that the flexible container 9 expands again, in order to facilitate the compression of the flexible container 9.

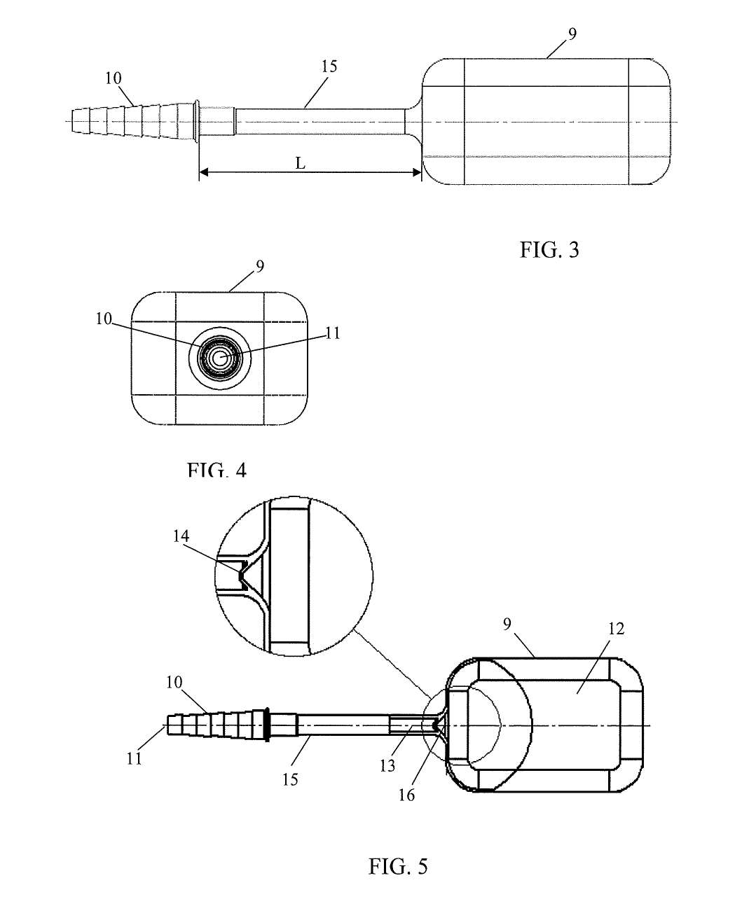

In one embodiment, as illustrated in FIG. 3, the connector 10 and the flexible container 9 are connected to each other via a tube 15. Thus, the lumen 13 runs through the tube 15, which extends between the connector 10 and the flexible container 9. The length L of the tube 15 is less than 50 mm, and preferably 35 mm. However, the connector 10 may, as a constructional variation, be attached directly to the flexible container 9.

The diameter of the lumen may be approximately 10 mm. The diameter of the opening 11 may be approximately 6 mm.

In FIG. 4, a front view of the flushing device 8 according to the present invention, is shown. As can be seen the flexible container 9 is essentially box-shaped, i.e the flexible container 9 may have a rectangular cross-section. The box-shape is advantageous since it makes the container 9 easy to grip. However, the flexible container 9 may have other shapes, such as spherical or similar.

In FIG. 5 it is illustrated that the flexible container 9 comprises an outlet port 16 being connected the lumen 13, and that the non-return valve 14 is arranged adjacent to the outlet port 16. The non-return valve 14 may alternatively be arranged closer to or adjacent to the opening 11 in the connector 10.

The connector 10 may comprise a removable tip portion (not shown) which is adapted to seal the flushing device 8. This is particularly advantageous when having a pre-filled compartment 12.

In FIG. 2-5, the connector 10 is a male connector. However, the connector 10 may be a female connector, or any other suitable connector adapted to be connected to a catheter.

As illustrated in FIG. 5, the non-return valve 14 is a non-return flap valve. As a constructional variation, the non-return valve 14 may be a ball check valve, or any other suitable non-return valve, such as a circular rubber membrane provided with an aperture or an opening with a flap, in the centre. When pressure is exerted on the outside of the flexible container 9, the flap will open by means of the liquid, thereafter, when there is no pressure, the flap will fall back such that the non-return valve is closed. Alternatively, the membrane may be provided with two openings comprising flaps.

The flexible container 9 is preferably made from polythene, or any other soft material having similar flexible or elastic material properties.

The connector 10 of the flushing device 8 is adapted to fit all urinary catheters worldwide since the catheters are manufactured according to a universal standard, and in the shown embodiment it is of the male type having tapered conical segments for providing a friction fit in the catheter coupling.

Other conceivable types of connectors would be Luer-Lok® connectors, bayonet type couplings and screw couplings just to mention a few.

The non-return valve 14 is opened when pressure is exerted on the flexible container 9. Liquid is then allowed to pass through the lumen 13 from the compartment 12 to the opening 11 and exit through the opening 11.

There are several alternative areas of use for the flushing device 8. For example, the flushing device 8 may be used directly or in a modified form, when flushing out a urinary catheter on urinary retention, when flushing out a bladder, or when catheterising animals using the same method as humans.

In another aspect the invention provides a new method for urinary catheterization which is performed as follows:
1. Wash hands with soap and warm water
2. Attach the flushing device 8 to the catheter.
3. Rinse the catheter carefully with fluid while it remains in its protective packaging in order to moisten the outside of the catheter
4. The penis should be lying on a flat surface
5. Moisten the urethra with a few drops of liquid from the flushing device 8 via the catheter
6. Insert the catheter into the urethra, holding the penis and the catheter and introduce the liquid
7. Then insert the catheter about 5 cm further. Never force it.
8. Continue to rinse and to feed in the catheter until it is fully inserted
9. Remove the flushing device 8 and wait for spontaneous flow of urine. Allow the urine to run out into a kidney dish.
10. The catheter balloon should be filled according to the manufacturer's instructions.
   Thanks to the rinsing method used with the flushing device 8, special catheters or trained experts are seldom needed.

In still another aspect of the invention there is provided a kit comprising at least a catheter and the new flushing device 8, the kit components being packaged in a sterile enclosure. The kit may further comprise an insertion tray/container, a sterile packaging, 2 drapes—moisture proof, underpad fenestrated, powder free gloves, prefilled syringe with 10 cc sterile water for cuffing catheter balloon, Rayon balls five, compress 10×10 cm two and forceps for single us.

The flushing device 8 is suitably prefilled with a sterile physiological saline, and the kit components are packaged in a sterile enclosures, suitably a plastic bag.

The present invention is not limited to the above-described preferred embodiments. Various alternatives, modifications and equivalents may be used. Therefore, the above embodiments should not be taken as limiting the scope of the invention, which is defined by the appending claims.

1-10. (canceled)

11. Flushing device (8) usable in the catheterization of a bladder, comprising:
a flexible container (9) defining a compartment (12) adapted to be filled with a liquid,
a connector (10) attached to the container (9), the connector (10) being adapted to be connected to a catheter, and said connector (10) being provided with an opening (11); wherein
said flexible container (9) is interconnected with said connector (10) via a lumen (13), such that liquid can be passed through said connector (10) in said lumen (13) from said flexible container (9) and exit said opening (11), and further comprising
a non-return valve (14) arranged in said lumen (13).

12. Flushing device according to claim 11, wherein said connector (10) and said flexible container (9) are connected to each other via a tube (15).

13. Flushing device according to claim 12, wherein the length L of the tube (15) is less than 50 mm, and preferably 35 mm.

14. Flushing device according to claim 11, wherein said connector (10) is attached directly to said flexible container (9).

15. Flushing device according to claim 11, wherein said flexible container (9) comprises an outlet port (16) in fluid communication with said lumen (13), and wherein said non-return valve (14) is arranged adjacent to said outlet port (16).

16. Flushing device according to claim 11, wherein said connector (10) is a male connector.

17. Flushing device according to claim 11, wherein said non-return valve (14) is a non-return flap valve.

18. Flushing device according to claim 11, wherein said non-return valve (14) is a ball check valve.

19. Kit comprising at least a catheter and a flushing device according to claim 11, the kit components being packaged in a sterile enclosure.

20. Kit according to claim 19, wherein the flushing device (8) is filled with a sterile flushing liquid.

21. A method of inserting a catheter in the urethra of a male patient, comprising the steps of:
providing a urinary catheter;
attaching a flushing device according to claim 11, with a flexible container filled with liquid, to the catheter;
inserting the catheter into the urethra;
causing liquid to flow out of the container and into the catheter thereby providing flushing, whereby the liquid acts as a lubricant, and feeding in the catheter until it is fully inserted in the urethra; and
removing the flushing device.

22. The method according to claim 21, further comprising rinsing the catheter carefully with fluid from the flushing device, while it remains in its protective packaging in order to moisten the outside of the catheter.

23. The method according to claim 21, further comprising moistening the urethra with a few drops of liquid from the flushing device via the catheter before inserting it into the urethra.

24. The method according to claim 21, further comprising after the insertion of the catheter, feeding the catheter about 5 cm further in the urethra.

25. The method according to claim 21, further comprising awaiting spontaneous flow of urine when the flushing device has been removed.

26. The method according to claim 25, further comprising filling the catheter balloon according to the manufacturer's instructions after ascertaining the spontaneous flow of urine.

27. The method according to claim 21, wherein the flushing liquid is a sterile physiological saline.

28. Flushing device according to claim 12, wherein said flexible container (9) comprises an outlet port (16) in fluid communication with said lumen (13), and wherein said non-return valve (14) is arranged adjacent to said outlet port (16).

29. Flushing device according to claim 13, wherein said flexible container (9) comprises an outlet port (16) in fluid communication with said lumen (13), and wherein said non-return valve (14) is arranged adjacent to said outlet port (16).

30. Flushing device according to claim 14, wherein said flexible container (9) comprises an outlet port (16) in fluid communication with said lumen (13), and wherein said non-return valve (14) is arranged adjacent to said outlet port (16).
